(19) United States
(12) Patent Application Publication
(10) Pub. No.: US 2012/0277462 A1
(43) Pub. Date: Nov. 1, 2012

(54) Solventless Reaction Process

(75) Inventors: Thomas Edward Enright, Tottenham (CA); Pouneh Salehi, Burlington (CA); Kentaro Morimitsu, Mississauga (CA)

(73) Assignee: Xerox Corporation, Norwalk, CT (US)

(21) Appl. No.: 13/095,015

(22) Filed: Apr. 27, 2011

(51) Int. Cl. C07C 67/08

(52) U.S. Cl. 560/180

(57) ABSTRACT

A process including reacting at least one organic acid with at least one compound of the formula R—OH, in the presence of an optional catalyst, in a reaction mixture wherein the reaction mixture is substantially free of solvent, to form a reaction product, wherein the reaction product is an ester of citric acid or an ester of tartaric acid; optionally, heating the reaction mixture; and optionally, isolating the reaction product.

### RELATED APPLICATIONS

Commonly assigned U.S. patent application Ser. No. ______ (Attorney Docket number 20100007, entitled “Next-Generation Solid Inks From Novel Oxazoline Components, Developed for Robust Direct-to-Paper Printing”), filed concurrently herewith, is hereby incorporated by reference herein in its entirety.

Commonly assigned U.S. patent application Ser. No. ______ (Attorney Docket number 20100008, entitled “Oxazoline Derivatives: Novel Components for a Next-Generation Robust Solid Ink”), filed concurrently herewith, is hereby incorporated by reference herein in its entirety.

Commonly assigned U.S. patent application Ser. No. ______ (Attorney Docket number 20100868, entitled “Solid Ink Compositions Comprising Amorphous Esters of Citric Acid”), filed concurrently herewith, is hereby incorporated by reference herein in its entirety.

Commonly assigned U.S. patent application Ser. No. ______ (Serial Number not yet assigned, Attorney Docket number 20101076-US-NP, entitled “Print Process For Phase Separation Ink”), filed concurrently herewith, is hereby incorporated by reference herein in its entirety.

Commonly assigned U.S. patent application Ser. No. ______ (Attorney Docket number 20101094, entitled “Phase Change Inks and Methods of Making the Same”), filed concurrently herewith, is hereby incorporated by reference herein in its entirety.

Commonly assigned U.S. patent application Ser. No. ______ (Attorney Docket number 20101139, entitled “Phase Change Ink Components and Methods of Making the Same”), filed concurrently herewith, is hereby incorporated by reference herein in its entirety.

Commonly assigned U.S. patent application Ser. No. ______ (Attorney Docket number 20101140, entitled “Solid Ink Compositions Comprising Amorphous Esters of Tartaric Acid”), filed concurrently herewith, is hereby incorporated by reference herein in its entirety.

Commonly assigned U.S. patent application Ser. No. ______ (Attorney Docket number 20101141, entitled “Solid Ink Compositions Comprising Crystalline Esters of Tartaric Acid”), filed concurrently herewith, is hereby incorporated by reference herein in its entirety.

Commonly assigned U.S. patent application Ser. No. ______ (Attorney Docket number 20101142, entitled “Phase Change Inks and Methods of Making the Same”), filed concurrently herewith, is hereby incorporated by reference herein in its entirety.

Commonly assigned U.S. patent application Ser. No. ______ (Attorney Docket number 20101266, entitled “Solid Ink Compositions Comprising Crystalline-Amorphous Mixtures”), filed concurrently herewith, is hereby incorporated by reference herein in its entirety.

Commonly assigned U.S. patent application Ser. No. ______ (Attorney Docket number 20101286, entitled “Solid Ink Compositions Comprising Crystalline-Amorphous Mixtures”), filed concurrently herewith, is hereby incorporated by reference herein in its entirety.

Commonly assigned U.S. patent application Ser. No. ______ (Serial Number not yet assigned, Attorney Docket number 20101591-US-NP, entitled “Phase Change Ink”), filed concurrently herewith, is hereby incorporated by reference herein in its entirety.

### BACKGROUND

Disclosed herein is a solventless reaction process for preparing an amorphous component for solid or phase change ink. The process includes reacting at least one organic acid with at least one compound of the formula R—OH in the presence of an optional catalyst, in a reaction mixture wherein the reaction mixture is substantially free of solvent, to form a reaction product, wherein the reaction product is an ester of citric acid or an ester of tartaric acid; optionally, heating the reaction mixture; and optionally, isolating the reaction product.

Ink jetting devices are known in the art, and thus extensive description of such devices is not required herein. As described in U.S. Pat. No. 6,547,380, which is hereby incorporated by reference herein in its entirety, ink jet printing systems generally are of two types: continuous stream and drop-on-demand. In continuous stream ink jet systems, ink is emitted in a continuous stream under pressure through at least one orifice or nozzle. The stream is perturbed, causing it to break up into droplets at a fixed distance from the orifice. At the break-up point, the droplets are charged in accordance with digital data signals and passed through an electrostatic field that adjusts the trajectory of each droplet in order to direct it to a gutter for recirculation or a specific location on a recording medium. In drop-on-demand systems, a droplet is expelled from an orifice directly to a position on a recording medium in accordance with digital data signals. A droplet is not formed or expelled unless it is to be placed on the recording medium.

There are at least three types of drop-on-demand ink jet systems. One type of drop-on-demand system is a piezoelectric device that has as its major components an ink filled channel or passageway having a nozzle on one end and a piezoelectric transducer near the other end to produce pressure pulses. Another type of drop-on-demand system is known as acoustic ink printing wherein an acoustic beam exerts a radiation pressure against objects upon which it impinges. Thus, when an acoustic beam impinges on a free surface such as at the liquid/air interface of a pool of liquid from beneath, the radiation pressure which it exerts against the surface of the pool may reach a sufficiently high level to release individual droplets of liquid from the pool, despite the restraining force of surface tension. Focusing the beam on or near the surface of the pool intensifies the radiation pressure it exerts for a given amount of input power. Still another type of drop-on-demand system is known as thermal ink jet, or bubble jet, and produces high velocity droplets. The major components of this type of drop-on-demand system are an ink filled channel having a nozzle on one end and a heat generating resistor near the nozzle. Printing signals representing digital information originate an electric current pulse in a resistive layer within each ink passageway near the orifice or nozzle, causing the ink vehicle (usually water) in the immediate vicinity to vaporize almost instantaneously and create a bubble. The ink at the orifice is forced out as a propelled droplet as the bubble expands.

In a typical design of a piezoelectric ink jet device utilizing phase change or solid inks printing directly on a substrate or on an intermediate transfer member, such as the one described in U.S. Pat. No. 5,372,852, which is hereby incorporated by reference herein in its entirety, the image is applied by jetting appropriately colored inks during four to eighteen rotations (incremental movements) of a substrate (an image receiving member or intermediate transfer member) with respect to the ink jetting head, i.e., there is a small translation of the print head with respect to the substrate in between each rotation. This approach simplifies the print head design, and the small movements ensure good droplet registration. At the jet operating temperature, droplets of liquid ink are ejected from the printing device and, when the ink droplets contact the surface of the recording substrate, either directly or via an intermediate heated transfer belt or drum, they quickly solidify to form a predetermined pattern of solidified ink drops.

Thermal ink jet processes are well known and are described, for example, in U.S. Pat. Nos. 4,601,777, 4,251,824, 4,410,899, 4,412,224 and 4,532,530, the disclosures of each of which are hereby totally incorporated herein.

As noted, ink jet printing processes may employ inks that are solid at room temperature and liquid at elevated temperatures. Such inks may be referred to as hot melt inks or phase change inks. For example, U.S. Pat. No. 4,490,731, which is hereby incorporated by reference herein in its entirety, discloses an apparatus for dispensing solid ink for printing on a substrate such as paper. In thermal ink jet printing processes employing hot melt inks, the solid ink is melted by the heater in the printing apparatus and utilized (i.e., jetted) as a liquid in a manner similar to that of conventional thermal ink jet printing. Upon contact with the printing substrate, the molten ink solidifies rapidly, enabling the colorant to substantially remain on the surface of the substrate instead of being carried into the substrate (for example, paper) by capillary action, thereby enabling higher print density than is generally obtained with liquid inks. Advantages of a phase change ink in ink jet printing are thus elimination of potential spillage of the ink during handling, a wide range of print density and quality, minimal paper cockle or distortion, and enablement of indefinite periods of nonprinting without the danger of nozzle clogging, even without capping the nozzles.

Solid inks for piezoelectric ink jet printing have been designed to successfully print in a transfix mode wherein the ink is jetted onto an intermediate transfer drum. In the transfix printing process, the ink cools from the jetting temperature (broadly, from about 75° C. and to no higher than about 180° C., and typically from about 110° C. to about 140° C.) to the drum temperature (typically from about 50° C. to about 60° C.), and, subsequently, as a substantially solid phase, the ink is pressed into a paper substrate. Such a process provides a number of advantages including vivid images, economy of jet use, and substrate latitude among porous papers. However, such ink designs can present problems when applied to coated papers. In general, the ink and the print process can fail to provide sufficient image durability in response to paper handling stresses such as scratch, fold and rub stresses.

Currently available phase change or solid ink printing processes are suitable for their intended purposes. However, a need remains for amorphous materials to provide certain characteristics to the printed ink, such as tack and robustness. Current processes for preparing amorphous material require use of a large amount of solvent and long reaction times, sometimes in excess of 45 hours. A further need remains for an improved process for preparing phase change or solid ink components that is cost effective, environmentally friendly, and efficient.

The appropriate components and process aspects of the each of the foregoing U.S. Patents and Patent Publications may be selected for the present disclosure in embodiments thereof. Further, throughout this application, various publications, patents, and published patent applications are referred to by an identifying citation. The disclosures of the publications, patents, and published patent applications referenced in this application are hereby incorporated by reference into the present disclosure to more fully describe the state of the art to which this invention pertains.

### SUMMARY

Described is a process comprising reacting at least one organic acid of the formula

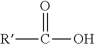

wherein R′ is (i) an alkyl group, which may be linear or branched, saturated or unsaturated, cyclic or acyclic, substituted or unsubstituted, and wherein heteroatoms either may or may not be present in the alkyl group, (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group, or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group; with at least one compound of the formula

R—OH

wherein R is selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group; and an optional catalyst; in a reaction mixture wherein the reaction mixture is substantially free of solvent, to form a reaction product; optionally, heating the reaction mixture; and optionally, isolating the reaction product; wherein the reaction product is a compound of the formula

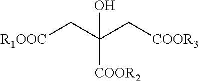

wherein R₁, R₂, and R₃ can be the same or different, and wherein R₁, R₂, and R₃ are each independently selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group; or wherein the reaction product is an ester of tartaric acid of the formula

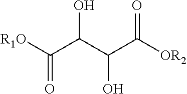

wherein a tartaric acid backbone is selected from L-(+)-tartaric acid, D-(−)-tartaric acid, DL-tartaric acid, mesotartaric acid, and mixtures thereof, and wherein R₁ and R₂ can be the same or different, and wherein R₁ and R₂ are each independently selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group.

### BRIEF DESCRIPTION OF THE DRAWINGS

FIG. 1 is graph illustrating percent conversion (y-axis) versus time (hours, x-axis) for a comparative reaction and a solventless reaction in accordance with the present process.

FIG. 2 is a bar graph illustrating milliliters of solvent per gram of reaction process for a comparative reaction and a solventless reaction in accordance with the present process.

FIG. 3 is bar graph illustrating reactor throughput (grams per liter) for a comparative reaction and a solventless reaction in accordance with the present process.

### DETAILED DESCRIPTION

A process is described comprising reacting at least one organic acid with at least one compound of the formula

R—OH

wherein R is (i) an alkyl group having from about 1 to about 40, or from about 1 to about 20, or from about 1 to about 10 carbon atoms, although the number of carbon atoms can be outside of these ranges, which may be linear or branched, saturated or unsaturated, cyclic or acyclic, substituted or unsubstituted, and wherein heteroatoms either may or may not be present in the alkyl group;

(ii) an aryl group, having from about 3 to about 40, or from about 6 to about 20, or from about 6 to about 10 carbon atoms, although the number of carbon atoms can be outside of these ranges, which may substituted or unsubstituted, and wherein heteroatoms either may or may not be present in the aryl group;

(iii) an arylalkyl group, having from about 4 to about 40, or from about 7 to about 20, or from about 7 to about 12 carbon atoms, although the number of carbon atoms can be outside of these ranges, which may be substituted or unsubstituted, wherein the alkyl portion of the arylalkyl group can be linear or substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group.

In another embodiment, the reaction product herein is an ester of tartaric acid of the formula

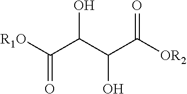

wherein a tartaric acid backbone is selected from L-(+)-tartaric acid, D-(−)-tartaric acid, DL-tartaric acid, mesotartaric acid, and mixtures thereof, and wherein R₁ and R₂ can be the same or different, and wherein R₁ and R₂ are each independently selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group.

The organic acid used for the process herein can be any suitable or desired organic acid. In embodiments, at least one organic acid comprising one, two, or three carboxylic acid groups is employed. In embodiments, the at least one organic acid comprises a carboxylic acid of the formula

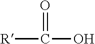

wherein R′ is (i) an alkyl group having from about 1 to about 40, or from about 1 to about 20, or from about 1 to about 10 carbon atoms, although the number of carbon atoms can be outside of these ranges, which may be linear or branched, saturated or unsaturated, cyclic or acyclic, substituted or unsubstituted, and wherein heteroatoms either may or may not be present in the alkyl group,

(ii) an aryl group having from about 3 to about 40, or from about 6 to about 20, or from about 6 to about 10 carbon atoms, although the number of carbon atoms can be outside of these ranges, which may be substituted or unsubstituted, and wherein heteroatoms either may or may not be present in the aryl group;

(iii) an arylalkyl group having from about 4 to about 40, or from about 7 to about 20, or from about 7 to about 12 carbon atoms, although the number of carbon atoms can be outside of these ranges, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group, or

(iv) an alkylaryl group having from about 4 to about 40, or from about 7 to about 20, or from about 7 to about 12 carbon atoms, although the number of carbon atoms can be outside of these ranges, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group.

In certain embodiments, the at least one organic acid is selected from the group consisting of acetic acid, propanoic acid, butanoic acid, pentanoic acid, citric acid, hexanoic acid, heptanoic acid, octanoic acid, decanoic acid, dodecanoic acid, tridecanoic acid, lauric acid, stearic acid, tartaric acid, and mixtures and combinations thereof.

In a specific embodiment, the at least one organic acid is citric acid. In another embodiment, the at least one organic acid is tartaric acid.

In embodiments, the reaction comprises reacting one or more R—OH compounds, such as a compound of the formula R₁—OH, R₂—OH, R₃—OH, or a mixture thereof, wherein R₁, R₂, and R₃ are the same or different, and wherein R₁, R₂, and R₃ are each independently selected from the definitions for R in R—OH as described above, and wherein, in embodiments, R₁, R₂, and R₃ are each independently selected from an alkyl group having from about 1 to about 40 carbon atoms; an aryl group having from about 3 to about 40 carbon atoms; an alkylaryl group having from about 4 to about 40 carbon atoms; and an arylalkyl group having from about 4 to about 40 carbon atoms.

In certain embodiments, R—OH is selected from the group consisting of

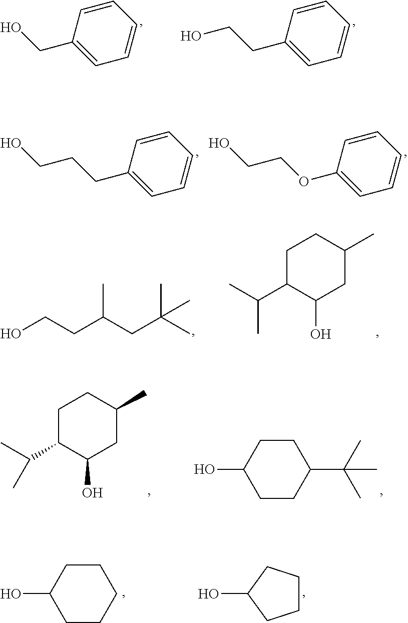

and mixtures thereof.

The organic acid and the alcohol can be provided in any desired or effective amounts. In one embodiment, the carboxylic acid and alcohol are provided in a 1:1 ratio of organic acid to alcohol although the ratio can be outside of this range.

The present process can be carried out at any suitable or desired temperature. In embodiments, heating the reaction mixture comprises heating to a temperature of from about 40 to about 250° C., or from about 90 to about 205° C., or from about 130 to about 180° C., although not limited to these ranges. In a specific embodiment, the reaction can be carried out at a temperature of about 170° C. In embodiments, the reaction temperature profile can be selected to increase the reaction rate.

The reaction can be heated for any suitable or desired amount of time. In embodiments, heating the reaction mixture comprises heating for a period of from about 1 to about 48 hours, or from about 4 to about 30 hours, or from about 6 to about 10 hours, although not limited to these ranges.

In a specific embodiment, heating the reaction mixture comprises heating to a temperature of from about 40 to about 250° C. for a period of from about 1 to about 48 hours. In another specific embodiment, heating the reaction mixture comprises heating to a temperature of from about 165 to about 175° C. for a period of from about 25 to about 30 hours. In yet another specific embodiment, heating the reaction mixture comprises heating to a temperature of 170° C. for a period of from about 27 hours.

Any suitable or desired catalyst can be used for the present reaction process. Examples of suitable catalysts include, but are not limited to, those selected from the group consisting of sulfuric acid, phosphoric acid, hydrochloric acid, p-toluenesulfonic acid, zinc chloride, magnesium chloride, zinc acetate, magnesium acetate, dibutyl tin laurate, and butylstannoic acid, and mixtures and combinations thereof. In a specific embodiment, the catalyst can be selected from the Fascat® series of catalysts available from Arkema, Inc., such as Fascat® 4100. The catalyst can be selected in any effective amount. For example, the catalyst can be present in an amount of from about 0.01 to about 1 percent by weight of the reaction mixture, although not limited to this range.

The process may include additional process steps. The process can further comprise steps of cooling and isolating the product which steps can be performed according to the knowledge of a person having ordinary skill in the art. Various techniques for these processing steps are known in the chemical arts.

In embodiments, the process comprises cooling the reaction mixture to room temperature and treating the reaction mixture with a solvent. While the present reaction process comprises a solventless reaction process, solvent can be used for downstream processing. For example, the final resin product can be discharged out of the reaction vessel and into a minimum amount of solvent to facilitate mixing with washing solutions, to facilitate material transfer between vessels, and the like. In embodiments, the process comprises cooling the reaction mixture to room temperature and treating the reaction mixture with an organic solvent selected from the group consisting of pentane, hexane, cyclohexane, heptane, octane, nonane, decane, undecane, dodecane, tridecane, tetradecane, toluene, xylene, benzene, and mesitylene. The amount of wash solvent is minimal. In embodiments, the amount of wash solvent is from about 0 to about 2 milliliters, or from about 0.5 to about 1.5 milliliters, or from about 0.75 to about 1 milliliter of wash solvent per gram of reaction product.

In embodiments, the process herein comprises treating the reaction mixture with a solvent, wherein the total amount of solvent used is less than about 1.5 milliliters of solvent per gram of reaction product, or wherein the total amount of solvent used is less than about 1 milliliter of solvent per gram of reaction product. In further embodiments, the process herein comprises cooling the reaction mixture to room temperature and treating the reaction mixture with a solvent; and wherein the total amount of solvent used is less than about 1.5 milliliters of solvent per gram of reaction product or less than about 1 milliliter of solvent per gram of reaction product.

The process can include the removal of water, such as through evaporation or distillation. The process may further include any additional chemical synthesis steps according to the knowledge of a person having ordinary skill in the art.

In embodiments, the reaction product can be isolated by any suitable or desired method, such as by filtering the reaction product. The process can further comprise drying the reaction product. Drying can be performed by any suitable or desired method at any suitable or desired temperature. In embodiments, drying can be under vacuum. Drying can be performed at any suitable or desired temperature, such as from about 20 to about 250° C., or from about 30 to about 200° C., or from about 80 to about 120° C. for any suitable or desired amount of time, such as from about 0.1 to about 48 hours, or from about 1 to about 24 hours, or from about 6 to about 8 hours.

In one embodiment, the reaction product herein can be a compound of the formula

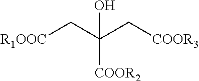

wherein R₁, R₂, and R₃ can be the same or different, and wherein R₁, R₂, and R₃ are each independently selected from (i) an alkyl group having from about 1 to about 40 carbon atoms, or from about 1 to about 20 carbon atoms, or from about 1 to about 10 carbon atoms, although the number of carbon atoms can be outside of these ranges, which may be linear or branched, saturated or unsaturated, cyclic or acyclic, substituted or unsubstituted, and wherein heteroatoms either may or may not be present in the alkyl group; (ii) an aryl group having from about 3 to about 40 carbon atoms, or from about 6 to about 20 carbon atoms, or from about 6 to about 10 carbon atoms, although the number of carbon atoms can be outside of these ranges, wherein heteroatoms either may or may not be present in the aryl group; (iii) an arylalkyl group having from about 4 to about 40 carbon atoms, or from about 7 to about 20 carbon atoms, or from about 7 to about 12 carbon atoms, although the number of carbon atoms can be outside of these ranges, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group having from about 4 to about 40 carbon atoms, or from about 7 to about 20 carbon atoms, or from about 7 to about 12 carbon atoms, although the number of carbon atoms can be outside of these ranges, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group.

In another embodiment, the reaction product herein can be an ester of tartaric acid of the formula

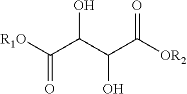

wherein the tartaric acid backbone is selected from L-(+)-tartaric acid, D-(−)-tartaric acid, DL-tartaric acid, mesotartaric acid, and mixtures thereof, and wherein R₁ and R₂ can be the same or different, and wherein R₁ and R₂ are each independently selected from R₁ and R₂ as described above.

In a specific embodiment, the reaction product is a compound of the formula

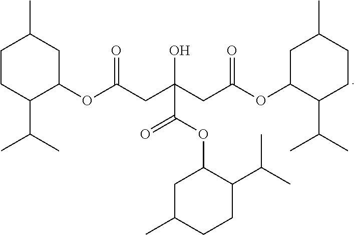

The present process enables preparation of an amorphous resin wherein the reaction step is solventless and an appropriate catalyst is employed to facilitate the reaction. The reaction process herein proceeds at a faster rate than previous solvent based reaction processes, and throughput is increased over previous solvent based reaction processes. Further, the present process is environmentally friendly due to reduction in overall solvent usage.

In embodiments herein, the reaction conversion percent is about 88 percent conversion in less than about 30 hours. In further embodiments herein, the reaction conversion percent is about 88 percent conversion in less than about 26 hours.

In further embodiments, the reaction throughput is about 350 grams of product per liter of reactor space.

### EXAMPLES

The following Examples are being submitted to further define various species of the present disclosure. These Examples are intended to be illustrative only and are not intended to limit the scope of the present disclosure. Also, parts and percentages are by weight unless otherwise indicated.

### Comparative Example 1

A solvent-based comparative reaction was carried out to produce an ester of citric acid in accordance with the following reaction scheme:

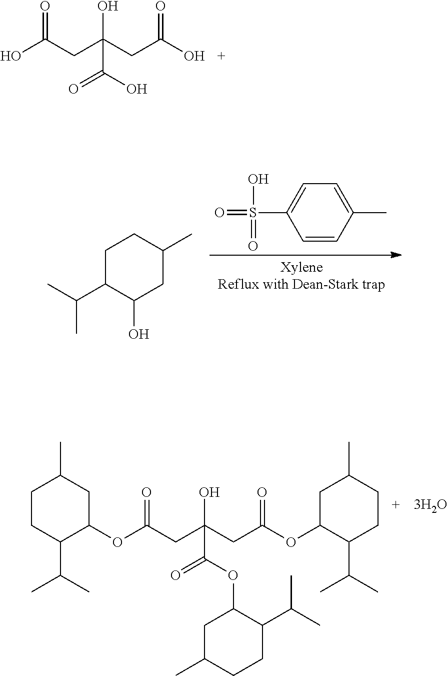

98.5 grams (0.5 mol) citric acid, 240.4 grams (1.5 mol) DL-menthol, and 1,230 milliliters of xylene were added to a 2 liter stainless steel Buchi reactor equipped with a Dean-Stark trap to give a suspension. 1.95 grams (0.01 mol) p-toluene sulfonic acid monohydrate were added and the mixture was refluxed for 45 hours with azeotropic removal of water. Reaction conversion was monitored by volume of water that was removed through the Dean-Stark trap. The reaction mixture was cooled to room temperature and washed with 10 weight percent aqueous sodium hydroxide (1×) and brine (2×) and then dried over MgSO₄. After filtration and removal of the solvent by vacuum distillation, the residue was dried under vacuum at 120° C. to obtain 250.5 grams (yield: 80%) of amorphous solid product.

### Example 2

A solventless reaction in accordance with the present disclosure was performed as follows. 287.4 grams (1.5 mol) citric acid, 701.3 grams (4.5 mol), and 0.9 grams Fascat® 4100 catalyst (available from Arkema, Inc.) were added to a 2 liter stainless steel Buchi reactor equipped with a Dean-Stark trap. The mixture was heated to 170° C. and held for 27 hours with removal of water through the Dean-Stark trap. The reaction mixture was cooled to room temperature and added to 750 milliliters of xylene for downstream processing. This solution was washed with 10 weight percent aqueous sodium hydroxide (1×) and brine (2×) and then dried over MgSO₄. After filtration and removal of the solvent by vacuum distillation, the residue was dried under vacuum at 120° C. for 8 hours to obtain 699.3 grams (yield: 77%) of amorphous solid product.

Example 2 demonstrated a solventless reaction process with minimal solvent usages for downstream processing. In embodiments, the final resin product of the solventless reaction process herein can be discharged out of the reactor and into a minimum amount of solvent to facilitate mixing with washing solutions and for facilitating material transfer between vessels. The process herein can include minimizing wash solvent volume alone or in combination with alternative purification methods to minimize or eliminate solvent use altogether.

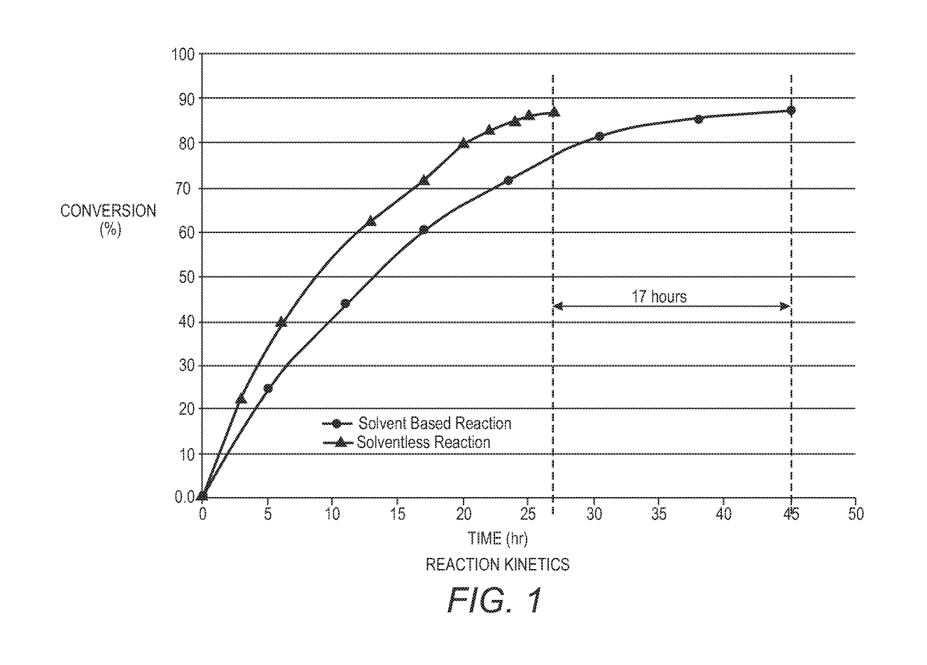

FIG. 1 illustrates reaction kinetics for the present solventless reaction process of Example 2 versus the solvent based reaction of Comparative Example 1. The graph of FIG. 1 provides conversion (%, y-axis) versus time (hours, x-axis) for the solvent based reaction of Comparative Example 1 versus the solventless reaction of Example 2. As can be seen in FIG. 1, the reaction kinetics are faster for the present solventless reaction process wherein the solvent is absent during the reaction.

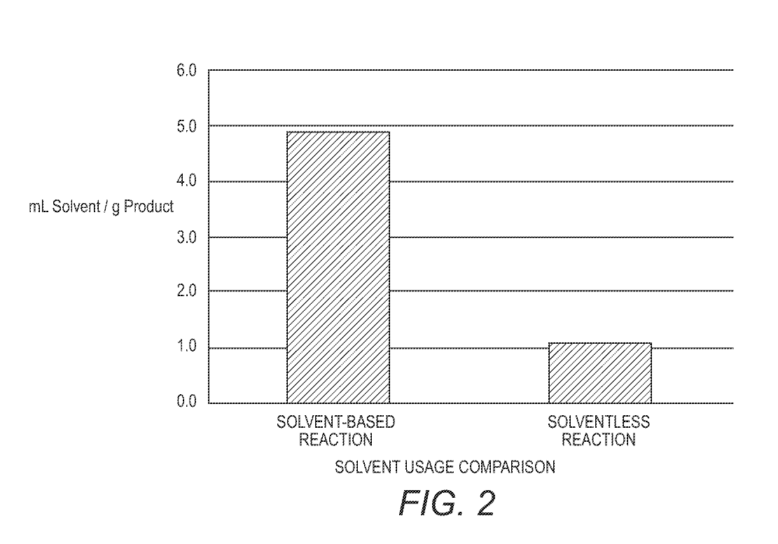

FIG. 2 illustrates milliliters of solvent per gram of reaction process for the comparative reaction of Comparative Example 1 and the solventless reaction of Example 2 in accordance with the present process. As illustrated in FIG. 2, the solventless reaction process provides for reduction or elimination of solvent usage wherein solvent usage, if at all, is limited to downstream washing operations.

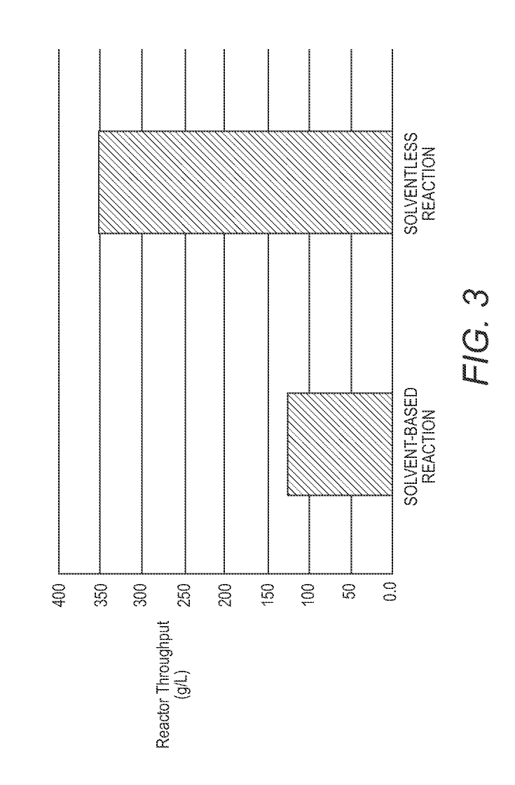

FIG. 3 illustrates reactor throughput (grams per liter) for the comparative reaction of Comparative Example 1 and the solventless reaction of Example 2 in accordance with the present process. The comparison of reactor throughput (grams of product per liter of reactor space) for the present solventless reaction versus the solvent based reaction shows how the present solventless reaction process enables, in embodiments, an approximately three-fold increase in throughput for a given reactor volume.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, shape, angle, color, or material.

1. A process comprising:
reacting at least one organic acid of the formula 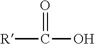
wherein R′ is (i) an alkyl group, which may be linear or branched, saturated or unsaturated, cyclic or acyclic, substituted or unsubstituted, and wherein heteroatoms either may or may not be present in the alkyl group, (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group, or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group;
with at least one compound of the formula
R—OH
wherein R is selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group; and
an optional catalyst;
in a reaction mixture wherein the reaction mixture is substantially free of solvent, to form a reaction product;
optionally, heating the reaction mixture; and
optionally, isolating the reaction product;
wherein the reaction product is a compound of the formula 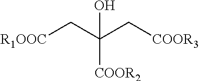
wherein R₁, R₂, and R₃ can be the same or different, and wherein R₁, R₂, and R₃ are each independently selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group; or
wherein the reaction product is an ester of tartaric acid of the formula 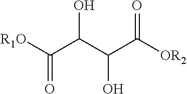
wherein a tartaric acid backbone is selected from L-(+)-tartaric acid, D-(−)-tartaric acid, DL-tartaric acid, mesotartaric acid, and mixtures thereof, and wherein R₁ and R₂ can be the same or different, and wherein R₁ and R₂ are each independently selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group.

2. The process of claim 1, wherein the at least one organic acid is citric acid.

3. The process of claim 1, wherein the at least one organic acid is tartaric acid.

4. The process of claim 1, wherein R—OH is selected from the group consisting of 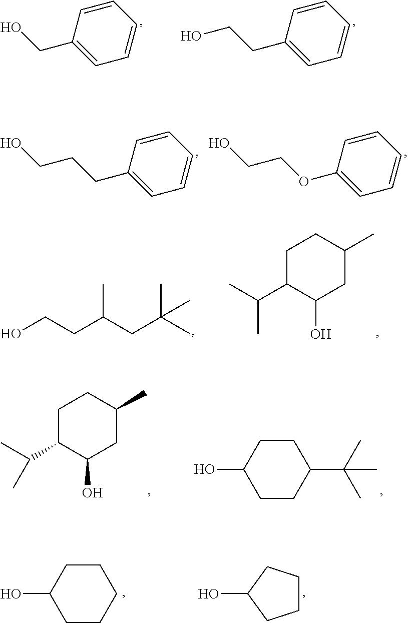
and mixtures thereof.

5. The process of claim 1, wherein R—OH is

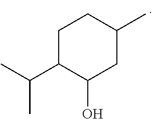

6. The process of claim 1, wherein the optional catalyst is selected from the group consisting of sulfuric acid, phosphoric acid, hydrochloric acid, p-toluenesulfonic acid, zinc chloride, magnesium chloride, zinc acetate, magnesium acetate, dibutyl tin laurate, and butylstannoic acid.

7. The process of claim 1, wherein heating the reaction mixture comprises heating to a temperature of from about 40 to about 250° C.

8. The process of claim 1, further comprising:
cooling the reaction mixture to room temperature and treating the reaction mixture with a solvent.

9. The process of claim 1, further comprising:
cooling the reaction mixture to room temperature and treating the reaction mixture with an organic solvent selected from the group consisting of pentane, hexane, cyclohexane, heptanes, octane, nonane, decane, undecane, dodecane, tridecane, tetradecane, toluene, xylene, benzene, and mesitylene.

10. The process of claim 1, further comprising:
cooling the reaction mixture to room temperature and treating the reaction mixture with a solvent; and
wherein the total amount of solvent used is less than about 1.5 milliliters of solvent per gram of reaction product.

11. The process of claim 1, further comprising:
treating the reaction mixture with a solvent, wherein the total amount of solvent used is less than about 1.5 milliliters of solvent per gram of reaction product.

12. The process of claim 1, further comprising:
treating the reaction mixture with a solvent, wherein the total amount of solvent used is less than about 1 milliliter of solvent per gram of reaction product.

13. The process of claim 1, wherein isolating the reaction product comprises filtering the reaction product.

14. The process of claim 1, further comprising drying the reaction product.

15. The process of claim 1, further comprising drying the reaction product at a temperature of from about 20 to about 250° C.

16. The process of claim 1, wherein the reaction product is a compound of the formula 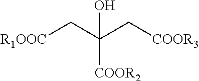
wherein R₁, R₂, and R₃ can be the same or different, and wherein R₁, R₂, and R₃ are each independently selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group.

17. The process of claim 1, wherein the reaction product is an ester of tartaric acid of the formula 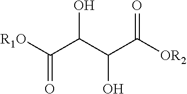
wherein a tartaric acid backbone is selected from L-(+)-tartaric acid, D-(−)-tartaric acid, DL-tartaric acid, mesotartaric acid, and mixtures thereof, and wherein R₁ and R₂ can be the same or different, and wherein R₁ and R₂ are each independently selected from (i) an alkyl group, which may be linear or branched, saturated or unsaturated, cyclic or acyclic, substituted or unsubstituted, and wherein heteroatoms either may or may not be present in the alkyl group; (ii) an aryl group, which may be substituted or unsubstituted, and wherein heteroatoms either may or may not be present in the aryl group; (iii) an arylalkyl group, which may be substituted or unsubstituted, wherein the alkyl portion of the arylalkyl group can be linear or branched, saturated or unsaturated, cyclic or acyclic, substituted or unsubstituted, and wherein heteroatoms either may or may not be present in either the aryl or the alkyl portion of the arylalkyl group; or (iv) an alkylaryl group, which may be substituted or unsubstituted, wherein the alkyl portion of the alkylaryl group can be linear or branched, saturated or unsaturated, cyclic or acyclic, substituted or unsubstituted, and wherein heteroatoms either may or may not be present in either the aryl or the alkyl portion of the alkylaryl group.

18. The process of claim 1, wherein the reaction product is a compound of the formula

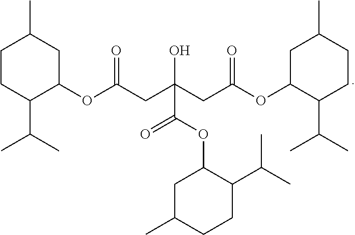

19. The process of claim 1, wherein the reaction conversion percent is about 88 percent conversion in less than about 30 hours.

20. The process of claim 1, wherein the reactor throughput is about 350 grams of product per liter of reactor space.
